(19) United States
(12) Patent Application Publication
(10) Pub. No.: US 2026/0351234 A1
(43) Pub. Date: Nov. 5, 2026

(54) METHOD AND DEVICE FOR OPTIMIZING ARRANGEMENT OF ONE-DIMENSIONAL ACOUSTIC DIFFUSE REFLECTION METASURFACE, AND STORAGE MEDIUM

(71) Applicant: Tongling University, Tongling (CN)

(72) Inventor: Ming Chen, Tongling (CN)

(21) Appl. No.: 19/380,864

(22) Filed: Nov. 5, 2025

(30) Foreign Application Priority Data
Apr. 30, 2025 (CN) 202510567416.X

(51) Int. Cl. G10K 11/20; B82Y 30/00; G06F 30/27; G06F 111/08; G06N 7/01

(52) U.S. Cl. CPC G10K 11/20; B82Y 30/00; G06F 30/27; G06N 7/01; G06F 2111/08

(57) ABSTRACT

A method for optimizing arrangement of a one-dimensional acoustic diffuse reflection metasurface is provided, which includes: initializing metasurface units and a unit arrangement corresponding to the metasurface units, calculating an acoustic pressure distribution of the metasurface units, and obtaining a standard deviation of a scattered acoustic pressure distribution; performing global search of a unit arrangement scheme by using MARKOV CHAIN MONTE CARLO (MCMC) annealing optimization to acquire an optimal arrangement of the metasurface units as an initial state for Q-Learning; applying Q-Learning reinforcement learning to further optimize the arrangement to obtain a new optimal unit arrangement scheme, thereby achieving more uniform scattering on the metasurface units of the new optimal unit arrangement scheme, and thus obtain an optimized metasurface arrangement; and calculating a scattered acoustic pressure corresponding to the optimized metasurface arrangement, and plotting a polar coordinate diagram of an optimized acoustic field distribution.

### CROSS-REFERENCE TO RELATED APPLICATION

This application claims the priority of Chinese Patent Application No. CN202510567416.X, filed Apr. 30, 2025, which is herein incorporated by reference in its entirety.

### TECHNICAL FIELD

The present disclosure relates to the field of acoustic metamaterials, and specifically relates to a method, device, and storage medium for optimizing arrangement of a one-dimensional acoustic diffuse reflection metasurface by integrating Markov Chain Monte Carlo (MCMC) and Q-Learning.

### BACKGROUND

Acoustic metasurfaces technology is used to design new metamaterials that can realize noise control, stealth technology and ultrasonic imaging. By designing fine unit structures on the subwavelength scale, acoustic metasurface can achieve extraordinary manipulation of acoustic waves, such as negative refraction, focusing, wave absorption and diffuse reflection. Among these, diffuse reflection metasurfaces offer the advantage of distributing incident acoustic energy uniformly in multiple directions, thereby mitigating energy concentration that may compromise noise suppression and echo control. Achieving high-performance diffuse reflection critically depends on the spatial arrangement of the metasurface unit structure.

In order to combine the various units in a reasonable way and thus make the scattered acoustic field uniformly distributed in space, existing technologies generally adopt traditional optimization methods such as genetic algorithms, simulated annealing algorithms, and particle swarm optimization. Although these methods can explore the solution space of metasurface arrangement to a certain extent, due to the discreteness and high nonlinearity of the arrangement problem itself, these methods are often prone to falling into local optimality. At the same time, a single global search strategy is difficult to take into account both global and local optimization requirements, resulting in unsatisfactory diffuse reflection effects.

Recently, the use of Markov Chain Monte Carlo (MCMC) methods has been introduced into metasurface design due to their superior global search capabilities. By employing stochastic perturbations along with probabilistic acceptance rules, MCMC can rapidly identify promising initial arrangement. Nevertheless, MCMC methods has certain shortcomings in local fine optimization. On the other hand, reinforcement learning approaches such as Q-Learning offer an adaptive framework capable of learning optimal policies through iterative interaction with the environment, thus enabling local fine-tuning and fine-grained adjustment. However, using reinforcement learning alone is easily affected by the large dimension of the state space and insufficient training samples, making it difficult to converge stably to the global optimal solution.

To address the limitations noted above, the present application proposes a method for optimizing the arrangement of a one-dimensional acoustic diffuse reflection metasurface that integrates global and local optimization capabilities.

### SUMMARY

The principal objective of the present disclosure is to provide a method for optimizing arrangement of a one-dimensional acoustic diffuse reflection metasurface. This is achieved by first applying an MCMC algorithm to perform a global search and acquire a preliminarily optimized arrangement. Subsequently, a Q-Learning algorithm is employed to locally refine this preliminary arrangement, thereby further reducing the standard deviation of the scattered acoustic field and enhancing the directional uniformity of the diffuse energy distribution. This approach effectively addresses the technical challenges identified in the background section.

To overcome the aforementioned issues, the present disclosure adopts the following technical solutions.

A method for optimizing arrangement of a one-dimensional acoustic diffuse reflection metasurface is provided, which includes:
initializing metasurface units and a unit arrangement corresponding to the metasurface units, calculating an acoustic pressure distribution of the metasurface units, and obtaining a standard deviation of a scattered acoustic pressure distribution;
performing global search of a unit arrangement scheme by using MCMC annealing optimization to acquire an optimal arrangement of the metasurface units as an initial state for Q-Learning;
applying Q-Learning reinforcement learning to further optimize the optimal arrangement to obtain a new optimal unit arrangement scheme, thereby achieving more uniform scattering on the metasurface units of the new optimal unit arrangement scheme, and thus obtain an optimized metasurface arrangement; and
calculating a scattered acoustic pressure corresponding to the optimized metasurface arrangement, and plotting a polar coordinate diagram of an optimized acoustic field distribution.

In an embodiment, the initializing metasurface units and a unit arrangement corresponding to the metasurface units, calculating an acoustic pressure distribution of the metasurface units, and obtaining a standard deviation of a scattered acoustic pressure distribution includes:
defining the metasurface units with a number of N, where each of the N metasurface units is selected from K predefined reflection angles {θ₁, θ₂, . . . , θ_{K}};
randomly initializing the unit arrangement S₀ corresponding to the N metasurface units, calculating the acoustic pressure distribution of the N metasurface units, and determining an objective function J as the standard deviation of the scattered acoustic pressure distribution, where the objective function J is expressed as follows:

$$J = \sqrt{\frac{1}{N} {\sum }_{i = 1}^{N} {( {P}_{i} - \overline{P} )}^{2}}$$

where Pᵢ presents a normalized acoustic pressure level at each angle, P̅ represents a mean acoustic pressure over all angles, N represents a number of discrete sampling points for scattering angles, and J indicates a scattering uniformity error, with a smaller value corresponding to a more uniform scattering distribution.

In an embodiment, the performing global search of a unit arrangement scheme by using MCMC annealing optimization to acquire an optimal arrangement of the metasurface units as an initial state for Q-Learning includes:
setting optimization hyperparameters, the optimization hyperparameters including a temperature T, an annealing coefficient α, and a maximum number of iterations MaxIter_MCMC;
adopting a Metropolis-Hastings algorithm to: randomly select a target metasurface unit from N metasurface units, and randomly select a target angle from the K predefined reflection angles, and modify a reflection angle of the target metasurface unit to the target angle to generate a new unit arrangement S_new;
after each iteration, updating the temperature by a formula T=α*T, and recording a current optimal solution; and
continuously updating the current optimal arrangement throughout an entire iteration process until T approaches 0 or the maximum number of iterations MaxIter_MCMC is reached.

In an embodiment, a process for generating a new unit arrangement S_new includes:
calculating a variation ΔJ of the objective function based on a formula expressed as follows:

ΔJ=J_{new}−J_{old}
where J_{new} and J_{old} represent variances of a metasurface scattered acoustic pressure before and after the change respectively; and
determining whether to accept the new unit arrangement S_new based on a Metropolis criterion, including:
in response to ΔE<0, which indicates the new unit arrangement S_new results in improved scattering uniformity, directly accepting the new unit arrangement S_new;
in response to ΔE≥0, determining whether to accept S_new based on a probability P=e−ΔJ/T, where T denotes an annealing parameter.

In an embodiment, the applying Q-Learning reinforcement learning to further optimize the optimal arrangement to obtain a new optimal unit arrangement scheme, thereby achieving more uniform scattering on the metasurface units of the new optimal unit arrangement scheme, and thus obtain an optimized metasurface arrangement includes:
defining a Q-value table Q(S, A), initialized to zero, a learning rate μ, a discount factor γ, an exploration rate ε, and a maximum number of Q-Learning iterations MaxIter_Q;
selecting a current state S_t, and in each training episode, for every metasurface unit in the current state S_t, and
adopting, by an agent, a ε-greedy policy to select actions, including:
with a probability ε, randomly altering a reflection angle of a designated metasurface unit to another candidate angle;
with a probability 1−ε, selecting an optimal action that corresponds to a highest Q-value in the Q-value table Q(S, A) under the current state; and
after selecting the optimal action, adjusting a corresponding unit in the unit arrangement to generate a new state S_(t+1) and the optimized metasurface arrangement S_best.

In an embodiment, the adjusting a corresponding unit in the unit arrangement to generate a new state S_(t+1) and the optimized metasurface S_best includes:
computing a scattering uniformity J(S_(t+1)) corresponding to the new state S_(t+1), and subsequently calculating a reward R_t based on a formula expressed as follows:

R_t=J_best−J(S_(t+1))
where J_best represents an optimal scattered acoustic pressure of the metasurface units;
where when J(S_(t+1))<J_best, which indicates R_t is positive, indicating that the scattering uniformity has improved in the new state S_(t+1), optimization is further needed;
updating a Q-value to obtain an updated Q-value, including:
updating an optimal solution S_best, in response to J(S_(t+1))<J_best, setting S_best=S_(t+1), and performing an iteration operation with the following formula:

Q(Sₜ,Aₜ)=Q(Sₜ,Aₜ)+μ·(Rₜ+γmax_{α′}Q(Sₜ₊₁,α′)−Q(Sₜ,Aₜ))
where (Sₜ, Aₜ) represents a state-action pair, Q(Sₜ, Aₜ) represents a current Q-value of the state-action pair; Rₜ represents an immediate reward for the current action; γ represents a discount factor, 0≤γ≤1, and γ is used to control importance of future rewards; μ represents a learning rate, which determines an updating magnitude of the Q-value; max_{α′}Q(Sₜ₊₁, α′) represents a maximum Q-value over all possible actions α′ in a next state S_(t+1); α′ represents an action under the next state S_(t+1); Rₜ+γmax_{α′}Q(Sₜ₊₁, α′) represents a target value; and Rₜ+γmax_{α′}Q(Sₜ₊₁, α′)−Q(Sₜ, Aₜ) represents a temporal difference error, reflecting a discrepancy between the target value and the current Q-value of the state-action pair (Sₜ, Aₜ); and
taking the maximum Q-value obtained after selecting the optimal action as the updated Q-value; and
repeating a Q-value update process until either a preset number of iterations is reached or a Q-table variation becomes smaller than a predefined threshold, and then outputting, by a system, the optimized metasurface arrangement S_best.

In another aspect, the present disclosure further discloses a non-transitory computer-readable storage medium storing a computer program, where when the computer program is executed by a processor, the processor executes the steps of the above method.

On the other hand, the present disclosure also discloses a computing device including a memory and a processor, where the memory stores a computer program that, and when the computer program is executed by the processor, the processor executes the steps of the above method.

Based on the above technical scheme, the present disclosure provides a method for optimizing the arrangement of a one-dimensional acoustic diffuse reflection metasurface. In comparison with conventional approaches, the disclosure offers the following advantages:
1. The present disclosure introduces the Markov Chain Monte Carlo (MCMC) algorithm in the early stage of optimization, which can play a role of global search, thereby quickly converging to a better unit arrangement structure and avoiding falling into the local optimum; Subsequently, Q-Learning reinforcement learning is employed to locally fine-tune the preliminary results in the later stage of optimization, which further reduces the standard deviation of the scattered acoustic field and achieve maximum uniformity of the diffuse scattered energy in all directions.
2. The present disclosure uses the Q value table combined with the scattering uniformity of the current arrangement to calculate the reward in the process of iterative selection of the unit arrangement state. This allows the Q value to be dynamically adjusted based on performance feedback. It can stably converge to the global optimal solution during the reinforcement learning process, making it easier to output the final optimized metasurface arrangement.

It should be noted that the features described in this section are not intended to define the essential or limiting characteristics of the present disclosure, nor to restrict its scope. Other technical features of the present disclosure will be further clarified in the following detailed description. Of course, the implementation of the present disclosure does not necessarily require achieving all the advantages listed above simultaneously.

### BRIEF DESCRIPTION OF DRAWINGS

The accompanying drawings, which form part of the present specification, are provided to facilitate better understanding of the present disclosure. The illustrative embodiments and their descripti⋅ons are intended to support interpretation of the present disclosure, rather than to impose limitations on its scope.

FIG. 1 illustrates a schematic diagram of the overall flow of the method proposed in the present disclosure.

FIG. 2 illustrates a schematic diagram of initial random arrangement metasurface acoustic pressure distribution in the present disclosure.

FIG. 3 illustrates a schematic diagram of the acoustic pressure distribution of the metasurface after MCMC-based optimization.

FIG. 4 illustrates a schematic diagram of the acoustic pressure distribution of the metasurface after combined optimization via MCMC and Q-Learning.

### DETAILED DESCRIPTION OF EMBODIMENTS

The following section provides a comprehensive and explicit description of the technical solution embodied in the present disclosure with reference to the accompanying drawings. It is evident that the embodiments described herein represent only a portion of all possible implementations and are not exhaustive. Where no conflict arises, the embodiments and features in the embodiments of the present disclosure can be combined with each other. All additional variants developed by persons skilled in the art, based on the disclosed embodiments without inventive contribution, shall fall within the scope of protection of the present disclosure.

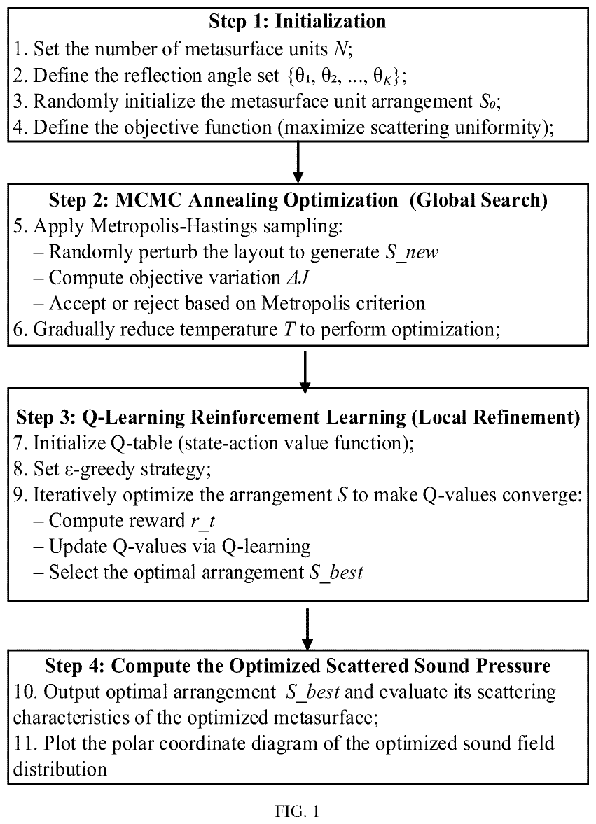

Reference is made to FIG. 1 through FIG. 4 for the embodiment.

As illustrated in FIG. 1, the proposed method for optimizing arrangement of a one-dimensional acoustic diffuse reflection metasurface includes the following steps.

### Step 1: Initialization

(1) N metasurface units are defined.
(2) Each unit selects its reflection angle from K predefined angles {θ₁, θ₂, . . . , θ_{K}}.
(3) The unit arrangement S₀ is Randomly initialized.
(4) The acoustic pressure distribution of the N metasurface units is calculated, and an objective function J is determined as the standard deviation of the scattered acoustic pressure distribution, where the objective function J is expressed as follows:

$$J = \sqrt{\frac{1}{N} {\sum }_{i = 1}^{N} {( {P}_{i} - \overline{P} )}^{2}}$$

where Pᵢ presents a normalized acoustic pressure level at each angle, P̅ represents a mean acoustic pressure over all angles, N represents a number of discrete sampling points for scattering angles, and J indicates a scattering uniformity error, with a smaller value corresponding to a more uniform scattering distribution.

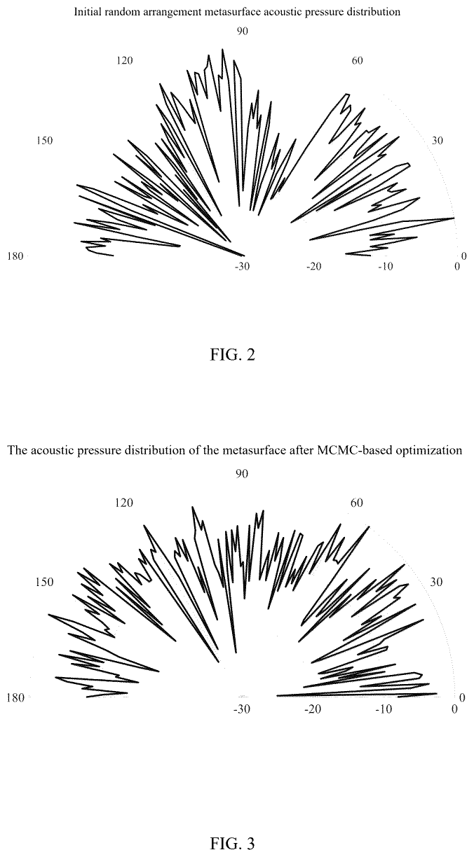

At this stage, the acoustic pressure distribution of the metasurface is shown in FIG. 2.

### Step 2: MCMC-Based Annealing Optimization (Global Search)

The primary objective is to explore various unit arrangement schemes and find a relatively optimal solution S_best, which will serve as the initial state for Q-Learning.
(5) The Metropolis-Hastings algorithm is applied to randomly select a unit i (from 1 to N) and modifying its reflection angle (randomly chosen from the K predefined angles), thereby generating a new arrangement S_new.

A variation ΔJ of the objective function is calculated based on the following formula: ΔJ=J_{new}−J_{old}; where J_{new} and J_{old} represent variances of a metasurface scattered acoustic pressure before and after the change respectively.

Whether to accept S_new is determined based on the Metropolis criterion:

when ΔE<0 (i.e., the new structural arrangement S_new yields improved scattering uniformity), then accept it directly; when ΔE≥0, the probability P=e−ΔJ/T is used to decide whether to accept S_new, where T denotes the annealing parameter. (6) After each iteration, the temperature is updated via T=α*T; and the current optimal solution is recorded. The optimal arrangement is continuously updated throughout the process until T approaches zero or MaxIter_MCMC is reached.

At this time, the acoustic pressure distribution of the metasurface is shown in FIG. 3.

### Step 3: Q-Learning Reinforcement Optimization (Local Search)

Objective: Based on S_best, the unit arrangement via reinforcement learning is further optimized to make the scattering more uniform.
(7) The Q-value table Q(S, A) (all entries set to zero), and a learning rate μ (controls update speed), a discount factor γ (weights future rewards), an exploration rate ε (balances exploration and exploitation), and a maximum number of Q-Learning iterations MaxIter_Q are defined.
(8) The current state S_t is selected (The current state S_t is the result S_best after the first stage MCMC optimization). In each iteration round, for each unit in S_t, the agent applies an ε-greedy policy: With probability ε, the unit's reflection angle is randomly changed to an alternative candidate (there are K angles in total); with probability 1−ε, an optimal action with a highest Q value in the corresponding state in the Q-value table is selected, that is, the action that maximizes the expected reward is selected.
(9) Once an action is selected, the corresponding unit in the arrangement is adjusted to generate the new state S_(t+1).

A reward R_t is calculated, which includes: computing a scattering uniformity J(S_(t+1)) and a reward as: Rₜ=J_{best}−J(Sₜ₊₁), when J(S_(t+1))<J_best then R_t is positive. That is, the uniformity of the new state increases and the standard deviation decreases, which encourages optimization.

The Bellman is applied to update to adjust the Q-value. When J(S_(t+1))<J_best, S_best=S_(t+1) is updated, and an iteration operation is performed based on the following formula:

Q(Sₜ,Aₜ)=Q(Sₜ,Aₜ)+μ·(Rₜ+γmax_{α′}Q(Sₜ₊₁,α′)−Q(Sₜ,Aₜ))
where (Sₜ, Aₜ) represents a state-action pair, Q(Sₜ, Aₜ) represents a current Q-value of the state-action pair; Rₜ represents an immediate reward for the current action; γ represents a discount factor, 0≤γ≤1, and γ is used to control importance of future rewards; μ represents a learning rate, which determines an updating magnitude of the Q-value; max_{a′}Q(Sₜ₊₁, α′) represents a maximum Q-value over all possible actions α′ in a next state S_(t+1); α′ represents an action under the next state S_(t+1); Rₜ+γmax_{α′}Q(Sₜ₊₁, α′) represents a target value; and Rₜ+γmax_{α′}Q(Sₜ₊₁, α′)−Q(Sₜ, Aₜ) represents a temporal difference error, reflecting a discrepancy between the target value and the current Q-value of the state-action pair (Sₜ, Aₜ).

The maximum Q-value obtained after selecting the optimal action is adopted as the updated Q-value.

Once a Q-value update is completed, proceed to the next iteration. A Q-value update process is repeated until either a preset number of iterations is reached or a Q-table variation becomes smaller than a predefined threshold. The training then terminates, and the system outputs the optimized metasurface arrangement S_best.

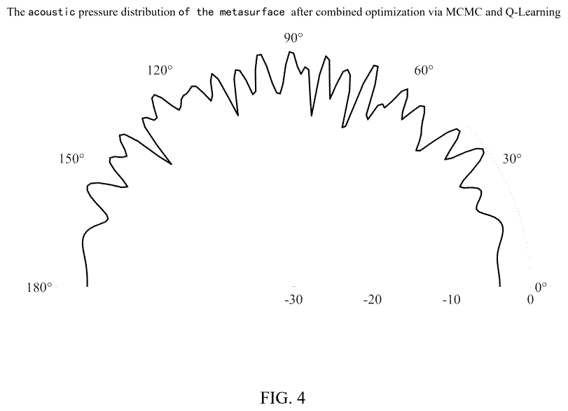

Step 4: Computation of Scattered Pressure after Optimization
(10) The optimal arrangement S_best is outputted, and calculate scattering characteristics of the optimized metasurface are calculated.
(11) A polar coordinate diagram of the acoustic field distribution of the optimized metasurface is plotted, as shown in FIG. 4.

To summarize, the optimization strategy proposed in this application follows a two-stage process:
Stage 1: Using the Markov Chain Monte Carlo (MCMC) method and the Metropolis-Hastings rule, a global search is performed based on the initial random arrangement to quickly converge to a better unit arrangement structure and avoid falling into the local optimum.
Stage 2: Based on the MCMC result, Q-Learning is applied to further optimize the arrangement and locally fine-tune the preliminary results to further reduce the standard deviation of the scattered sound field distribution and achieve maximum uniformity of the diffuse scattered energy in all directions.

In a specific implementation, the following parameters are applied to optimize the 1D metasurface arrangement.

### Parameter Settings

Number of units: N=40, Predefined reflection angles: K=5, specifically 15°, 30°, 45°, 60°, 80°.
Initial temperature: T=1.0, Annealing coefficient: α=0.9; Maximum MCMC iterations: MaxIter_MCMC=800.
Q-Learning parameters: learning rate α=0.05, discount factor γ=0.9, exploration rate ε=0.3, maximum Q-Learning iterations MaxIter_Q=2000.

The optimization process proceeds as follows: starting from a randomly initialized arrangement S₀, MCMC sampling is executed to obtain a preliminarily optimized arrangement S_init, which is then refined via Q-Learning to yield the final optimized arrangement S_best.

### Experimental Results

The standard deviation of the initial random arrangement is 5.69, and its corresponding acoustic pressure distribution is shown in FIG. 2. After MCMC optimization, the standard deviation decreases to 4.11, as illustrated in FIG. 3. Upon completing 2000 rounds of training combining MCMC and Q-Learning, the final optimized arrangement achieves a standard deviation of 2.44, with its acoustic field distribution presented in FIG. 4. The uniformity of the metasurface is improved by 57.12% compared with the initial random arrangement and by 40.63% compared with the single-stage MCMC optimization.

In another aspect, the present disclosure also discloses a non-transitory computer-readable storage medium that stores a computer program. When the computer program is executed by a processor, the steps described in the above method are performed.

In a further aspect, the present disclosure discloses a computing device including a memory and a processor. The memory stores a computer program. When the computer program is executed by the processor, the processor executes the steps of the above method.

In an additional embodiment, the present application further provides a computer program product containing instructions which, when run on a computer, enable the computer to perform any of the one-dimensional acoustic diffuse reflection metasurface optimization arrangement methods described in the above embodiments.

It is understood that the system provided in this embodiment of the present disclosure corresponds to the method provided by the embodiment of the present disclosure, and the relevant explanations, examples, and benefits may be referenced from the respective sections of the method.

This embodiment further discloses an electronic device including a processor, a communication interface, a memory, and a communication bus. The processor, the communication interface, and the memory communicate with one another via the communication bus.

The memory is configured to store a computer program.

The processor is used to execute the program stored in the memory to perform the above method.

The communication bus mentioned in the above electronic device can be a peripheral component interconnect standard bus or an extended industry standard architecture bus, etc. The communication bus can be divided into an address bus, a data bus, a control bus, etc.

The communication interface is used for communication between the above electronic device and other devices.

The memory may include random access memory or non-volatile memory such as at least one disk storage unit. Optionally, the memory may include one or more storage devices located remotely from the processor.

The processor may be a general-purpose processor such as a central processing unit or a network processor, or a specialized processor. It can also be a digital signal processor, an application-specific integrated circuit, a field programmable gate array or other programmable logic device, a discrete gate or transistor logic device, or a discrete hardware component.

It is also noted that the electronic device may include a terminal device, which may also be referred to as a terminal, user equipment, mobile station, or mobile terminal. The terminal device may be a mobile phone, smart television, wearable device, tablet computer, computer with wireless transceiving capability, virtual reality device, augmented reality device, wireless terminal for industrial control, autonomous driving, remote surgery, smart grid, transport safety, smart cities, smart homes, and the like. The embodiments of the present application do not limit the specific technology and specific device form adopted by the terminal device.

In the above embodiments, the present disclosure may be implemented entirely or in part via software, hardware, firmware, or any combination thereof. When implemented via software, it can be implemented in whole or in part in the form of a computer program product. The computer program product includes one or more computer instructions. When the computer program instructions are loaded and executed on a computer, the processes or functions described in the embodiments of the present application are generated in whole or in part. The computer may be a general purpose computer, a special purpose computer, a computer network, or other programmable device. These instructions may be stored in or transmitted from one computer-readable storage medium to another, such as via a website, computer, server, or data center, using wired (e.g., coaxial cable, fiber optic, DSL) or wireless (e.g., infrared, radio, microwave) transmission. The computer-readable storage medium can be any available medium that can be accessed by a computer, or a data storage device such as a server or data center that includes one or more available media. The medium may be magnetic (e.g., floppy disks, hard drives, magnetic tapes), optical, or semiconductor-based (e.g., solid-state drives).

The foregoing description represents the preferred embodiments of the present disclosure and is not intended to limit its scope. Any equivalent modifications, substitutions, or improvements made within the spirit and principles of the present disclosure shall fall within the scope of protection.

In addition, it should be noted that directional terms such as “upper”, “lower”, “left”, “right”, “front”, and “rear” used in the embodiments are merely to describe the relative spatial relationships under a given posture. These descriptions will correspondingly change if the specific posture changes.

Moreover, terms such as “first”, “second”, etc., used in the embodiments, are only used for descriptive purposes and cannot be understood as indicating or implying their relative importance or implicitly indicating the quantity of the indicated technical features. Therefore, features defined as “first” or “second” may explicitly or implicitly include at least one of the features. In addition, the term “and/or” appearing throughout the text includes three parallel options. For instance, “A and/or B” refers to A alone, B alone, or both A and B together. In the context of this application, the term “plurality” indicates two or more instances. Finally, the technical solutions between the various embodiments can be combined with each other, but they must be based on the fact that ordinary technicians in this field can implement them. When the combination of technical solutions is mutually contradictory or cannot be implemented, it should be deemed that such a combination of technical solutions does not exist and is not within the scope of protection required by the present disclosure.

1. A method for optimizing arrangement of a one-dimensional acoustic diffuse reflection metasurface, comprising:
initializing metasurface units and a unit arrangement corresponding to the metasurface units, calculating an acoustic pressure distribution of the metasurface units, and obtaining a standard deviation of a scattered acoustic pressure distribution;
performing global search of a unit arrangement scheme by using MARKOV CHAIN MONTE CARLO (MCMC) annealing optimization to acquire an optimal arrangement of the metasurface units as an initial state for Q-Learning;
applying Q-Learning reinforcement learning to further optimize the optimal arrangement to obtain a new optimal unit arrangement scheme, thereby achieving more uniform scattering on the metasurface units of the new optimal unit arrangement scheme, and thus obtain an optimized metasurface arrangement; and
calculating a scattered acoustic pressure corresponding to the optimized metasurface arrangement, and plotting a polar coordinate diagram of an optimized acoustic field distribution;
wherein the applying Q-Learning reinforcement learning to further optimize the arrangement to obtain a new optimal unit arrangement scheme, thereby achieving more uniform scattering on the metasurface units of the new optimal unit arrangement scheme, and thus obtain an optimized metasurface arrangement comprises:
defining a Q-value table Q(S, A), initialized to zero, a learning rate μ, a discount factor γ, an exploration rate ε, and a maximum number of Q-Learning iterations MaxIter_Q;
selecting a current state S_t, and in each training episode, for every metasurface unit in the current state S_t, adopting, by an agent, a ε-greedy policy to select actions, comprising:
with a probability ε, randomly altering a reflection angle of a designated metasurface unit to another candidate angle;
with a probability 1−ε, selecting an optimal action that corresponds to a highest Q-value in the Q-value table Q(S, A) under the current state; and
after selecting the optimal action, adjusting a corresponding unit in the unit arrangement to generate a new state S_(t+1) and the optimized metasurface arrangement S_best.

2. The method for optimizing the arrangement of the one-dimensional acoustic diffuse reflection metasurface according to claim 1, wherein the initializing metasurface units and a unit arrangement corresponding to the metasurface units, calculating an acoustic pressure distribution of the metasurface units, and obtaining a standard deviation of a scattered acoustic pressure distribution comprises:
defining the metasurface units with a number of N, wherein a reflection angle of each of the N metasurface units is selected from K predefined reflection angles {θ₁, θ₂, . . . , θ_{K}}; and
randomly initializing the unit arrangement S₀ corresponding to the N metasurface units, calculating the acoustic pressure distribution of the N metasurface units, and determining an objective function J as the standard deviation of the scattered acoustic pressure distribution, wherein the objective function J is expressed as follows:
$$J = \sqrt{\frac{1}{N} {\sum }_{i = 1}^{N} {( {P}_{i} - \overline{P} )}^{2}}$$
where Pᵢ presents a normalized acoustic pressure level at each angle, P̅ represents a mean acoustic pressure over all angles, N represents a number of discrete sampling points for scattering angles, and J indicates a scattering uniformity error, with a smaller value corresponding to a more uniform scattering distribution.

3. The method for optimizing the arrangement of the one-dimensional acoustic diffuse reflection metasurface according to claim 2, wherein the performing global search of a unit arrangement scheme by using MCMC annealing optimization to acquire an optimal arrangement of the metasurface units as an initial state for Q-Learning comprises:
setting optimization hyperparameters, the optimization hyperparameters including a temperature T, an annealing coefficient α, and a maximum number of iterations MaxIter_MCMC;
adopting a Metropolis-Hastings algorithm to: randomly select a target metasurface unit from N metasurface units, and randomly select a target angle from the K predefined reflection angles, and modify a reflection angle of the target metasurface unit to the target angle to generate a new unit arrangement S_new;
after each iteration, updating the temperature by a formula T=α*T, and recording a current optimal solution; and
continuously updating the current optimal arrangement throughout an entire iteration process until T approaches 0 or the maximum number of iterations MaxIter_MCMC is reached.

4. The method for optimizing the arrangement of the one-dimensional acoustic diffuse reflection metasurface according to claim 3, wherein a process for generating a new unit arrangement S_new comprises:
calculating a variation ΔJ of the objective function based on a formula expressed as follows:
ΔJ=J_{new}−J_{old}
where J_{new} and J_{old} represent variances of a metasurface scattered acoustic pressure before and after the change respectively; and
determining whether to accept the new unit arrangement S_new based on a Metropolis criterion, comprising:
in response to ΔE<0, which indicates the new unit arrangement S_new results in improved scattering uniformity, directly accepting the new unit arrangement S_new;
in response to ΔE≥0, determining whether to accept S_new based on a probability P=e−ΔJ/T, where T denotes an annealing parameter.

5. (canceled)

6. The method for optimizing the arrangement of the one-dimensional acoustic diffuse reflection metasurface according to claim 1, wherein the adjusting a corresponding unit in the unit arrangement to generate a new state S_(t+1) and the optimized metasurface S_best comprises:
computing a scattering uniformity J(S_(t+1)) corresponding to the new state S_(t+1), and subsequently calculating a reward R_t based on a formula expressed as follows:
R_t=J_best−J(S_(t+1))
where J_best represents an optimal scattered acoustic pressure of the metasurface units;
wherein when J(S_(t+1))<J_best, which indicates R_t is positive, indicating that the scattering uniformity has improved in the new state S_(t+1), optimization is further needed;
updating a Q-value to obtain an updated Q-value, comprising:
updating an optimal solution S_best; in response to J(S_(t+1))<J_best, setting S_best=S_(t+1), and performing an iteration operation with the following formula:
Q(Sₜ,Aₜ)=Q(Sₜ,Aₜ)+μ·(Rₜ+γmax_{α′}Q(Sₜ₊₁,α′)−Q(Sₜ,Aₜ))
where (Sₜ, Aₜ) represents a state-action pair, Q(Sₜ, Aₜ) represents a current Q-value of the state-action pair; Rₜ represents an immediate reward for the current action; γ represents a discount factor, 0≤γ≤1, and γ is used to control importance of future rewards; μ represents a learning rate, which determines an updating magnitude of the Q-value; max_{α′}Q(Sₜ₊₁, α′) represents a maximum Q-value over all possible actions α′ in a next state S(t+1); α′ represents an action under the next state S_(t+1); Rₜ+γmax_{α′}Q(Sₜ₊₁, α′) represents a target value; and Rₜ+γmax_{α′}Q(Sₜ₊₁, α′)−Q(Sₜ, Aₜ) represents a temporal difference error, reflecting a discrepancy between the target value and the current Q-value of the state-action pair (Sₜ, Aₜ); and
taking the maximum Q-value obtained after selecting the optimal action as the updated Q-value; and
repeating a Q-value update process until either a preset number of iterations is reached or a Q-table variation becomes smaller than a predefined threshold, and then outputting, by a system, the optimized metasurface arrangement S_best.

7. A computer-readable storage medium with a computer program stored thereon, wherein the computer program, when executed by a processor, is configured to perform the method according to claim 1.

8. A computer device, comprising a memory and a processor, wherein the memory stores a computer program which, when executed by the processor, is configured to perform the method according to claim 1.
